(19) United States
(12) Patent Application Publication
(10) Pub. No.: US 2015/0094266 A1
(43) Pub. Date: Apr. 2, 2015

(54) XYLOGLUCAN AND PROTEIN COMPOSITIONS FOR THE TREATMENT OF INTESTINAL DISORDERS

(71) Applicant: Novintethical Pharma, SA, Lugano (CH)

(72) Inventors: Marco Di Fulvio, Soriano nel Cimino (IT); Miguel Angel Alonso, Barcelona (ES); Michele Giuseppe Di Schiena, Robecco sul Naviglio (IT)

(73) Assignee: Novintethical Pharma, SA, Lugano (CH)

(21) Appl. No.: 14/566,546

(22) Filed: Dec. 10, 2014

Related U.S. Application Data
(63) Continuation-in-part of application No. 14/092,365, filed on Nov. 27, 2013.

(30) Foreign Application Priority Data
Jun. 11, 2013 (IT) MI2013A000960
Apr. 15, 2014 (IT) MI2014A000705

(51) Int. Cl. A61K 38/39; A61K 38/16; A61K 31/732; A61K 31/716; A61K 45/06

(52) U.S. Cl. CPC A61K 38/39; A61K 31/716; A61K 45/06; A61K 31/732; A61K 38/168
USPC 514/13.2; 514/17.2; 514/21.2

(57) ABSTRACT

Compositions include synergistic combinations of xyloglucans and vegetable or animal proteins that are useful in the treatment of intestinal disorders or of disorders originating from the intestinal system and transferred to other systems, such as the genitourinary system. The compositions include xyloglucans or an extract containing xyloglucans, and one or more animal or vegetable proteins. Methods for the treatment or prevention of intestinal disorders are provided, including prevention of diarrhoea.

### CROSS-REFERENCE TO RELATED APPLICATIONS

This is a continuation-in-part of co-pending U.S. application Ser. No. 14/092,365, filed Nov. 27, 2013, which claims priority to Italian Application No. MI2013A000960, filed Jun. 11, 2013. The present application also claims priority to Italian Application No. MI2014A000705, filed Apr. 15, 2014. Each of the foregoing applications are hereby incorporated by reference herein in their entirety.

### FIELD OF THE INVENTION

The invention refers to combinations that include synergistic combinations of xyloglucans and animal or vegetable proteins and to compositions for the treatment of intestinal disorders, in particular diarrhoeal diseases of various origin.

### BACKGROUND

Diarrhoea is an often debilitating and dangerous symptom of many gastrointestinal disorders, especially in children and the elderly. Acute diarrhoea is primarily caused by intestinal infections, but may also be due to the use of drugs, to radiation treatments and to other pathological conditions (diverticulitis, poisoning by heavy metals, intestinal ischemia, allergies or intolerances).

Acute diarrhoea due to infection is a serious problem in developing countries as it is believed to cause the death of at least 4 million children less than 5 years old annually.

Chronic diarrhoea is most often caused by irritable bowel syndrome, coeliac disease, or inflammatory bowel diseases (e.g., Crohn's disease, ulcerative colitis).

In the light of the various etiologies, different therapeutic options are available that are based on the administration of antibiotics/antibacterial agents, antispasmodic/anticholinergic agents, probiotics, and opioid receptor agonists. Some of these treatments must however be used with great caution as they do not act on the disease's causal process.

To overcome these negative effects, tannins complexed with animal proteins and gelatins, in particular with bovine gelatin, albumin, casein, and ovalbumin have long been proposed.

The use of such complexes in the treatment, for example, of diarrhoea in its various forms is described in EP 1764105, EP 2526939, EP 2361623 and US 20090062191. Gelatin tannate has long been commercially available in certain compositions or as a medical device for the treatment of acute diarrhoea.

Thus there arises the need for further therapeutic treatments capable of replacing or being administered alongside the ones available currently.

### SUMMARY OF THE INVENTION

In one embodiment, the invention provides pharmaceutical compositions comprising, as active ingredients, xyloglucans or xyloglucan-containing extracts and animal or vegetable proteins, combined with suitable excipients and possibly with other active ingredients useful for the treatment of intestinal disorders, in particular diarrhoeal diseases.

A composition for use in the prevention and treatment of intestinal disorders or of disorders originating in the intestinal system and transferred to other systems, is provided, said composition containing xyloglucans or extracts containing xyloglucans, and at least one animal or vegetable protein. One preferred protein is gelatin.

A method for prevention or treatment of intestinal disorders or of disorders originating in the intestinal system and transferred to other systems, is provided, said method comprising the steps of: providing a composition comprising xyloglucans or extracts containing xyloglucans, and at least one animal or vegetable protein; and administering an effective amount of the composition to a mammal in need thereof, said mammal having an intestinal disorder or a disorder originating in the gastro-intestinal system and transferred to other systems.

In a further embodiment, a method for the prevention of diarrhoea is provided, comprising the steps of: providing a composition comprising xyloglucans or extracts containing xyloglucans, and a gelatin; and orally administering an effective amount of the composition to a mammal in need thereof, whereby diarrhoea, or symptoms thereof, are prevented.

### BRIEF DESCRIPTION OF THE DRAWINGS

FIG. 1 depicts the difference in water secretion in isolated intestinal jejunal loops 2 hrs after cholera toxin (CT) challenge in anesthetized rats, where either xyloglucan (XG) or a combination of xyloglucan (XG) and gelatin (P) were orally administered 6 hrs before CT challenge.

FIG. 2 depicts the difference in water secretion in isolated intestinal jejunal loops 2 hrs after cholera toxin (CT) challenge in anesthetized rats, where either gelatin (Gel) was orally administered 6 hrs before CT challenge, or a combination of xyloglucan and gelatin (XG-Gel) was orally administered 12 hrs before CT challenge.

### DETAILED DESCRIPTION

Surprisingly, it has now been found that combinations of xyloglucans with vegetable or animal proteins compatible with oral administration in humans are particularly effective in the treatment and prevention of diarrhoea and other infectious and/or inflammatory intestinal disorders. Xyloglucans are believed to have a film-forming effect on intestinal mucosa that can decrease the permeability of the tight junctions of the intestinal mucosa and therefore impede the entry of the pathogens responsible for acute intestinal infections. The film-forming effect is not affected by pH changes.

In a principal embodiment the invention therefore provides pharmaceutical compositions that have, as active ingredients, xyloglucans or extracts that contain them in combination with vegetable or animal proteins, in addition to suitable excipients, and other optional active ingredients. In an embodiment, the compositions are useful for the prevention and treatment of intestinal and genitourinary disorders.

Xyloglucans are molecules composed of a linear skeleton of β-1,4-glucans with short lateral branches. These branches can bind to one another (and other polar, hydrophilic molecules) due to xylose bonded to oxygen at the 6-position of the sugar. These branched side chains may also contain other sugars such as arabinose and fucose.

Xyloglucans belong to the hemicellulose family associated with the cellulose within the cell wall of higher plants. A particularly rich source of xyloglucans is the seed of the tamarind (Tamarindus indica), a tropical tree native to East Africa.

Extracts of tamarind seeds rich in xyloglucans are known and have been used in the medical field mainly as viscosifying agents in ophthalmic compositions or uses (U.S. Pat. No. 6,056,950), mucoadhesive agents (WO2006/131262), tear substitutes (WO2009/044423), anti-infective agents (WO2011/147767) and anti-inflammatory agents (WO2011/147768).

The xyloglucans extracted from Tamarindus indica are commercially available, for example from Indena (Italy) (Xilogel®) and from DSP Gokyo Food & Chemical (Japan) (Glyloid®). The average molecular weight is between 400,000 and 650,000 daltons.

As used herein, the terms “vegetable or animal proteins” (e.g. compatible with oral administration) means proteins such as, but not limited to, gelatin, albumin, ovalbumin, casein, pea protein, and soy protein. Gelatin and pea protein are preferred. Gelatin is particularly preferred. Further, the term “protein” may be used in a generic way to indicate a combination, mixture, or blend of one or more vegetable or animal proteins.

A useful weight ratio between xyloglucan and protein is between about 1:0.5 and about 1:30. Stated in an alternative way, a useful weight ratio between xyloglucan and at least one protein is between about 1:0.5 and about 1:30. The combination of xyloglucan and protein covered by the invention constitutes the active ingredient of certain oral pharmaceutical formulations.

Examples of suitable forms of administration may include capsules, tablets, solutions, suspensions, granules, gels, and the like. One useful product containing a combination of xyloglucan (100 mg) and bovine/porcine gelatin is TASECTAN PLUS available from Novintethical Pharma, Sagl (Lugano, Switzerland), including gelatin dosages of 125 mg (pediatric) and 250 mg (adult).

Examples of other active ingredients and/or excipients with which the xyloglucans and protein (i.e. one or more proteins) can be combined include: antibiotics, antiperistaltic agents, steroidal or non-steroidal anti-inflammatory agents, compounds for the treatment of gastrointestinal bloating (simethicone and the like), electrolytes, mesalazine, sucralfate, natural and synthetic polysaccharides such as pectins, chitosan (animal or vegetable), hyaluronic acid, Guar gum, xanthan gum, celluloses and hemicelluloses (including derivatives such as for example hydroxypropyl cellulose), carrageenans, carbomers, crosslinking/polymerizing compounds such as ferulic acid; polyphenols, such as polyphenols from galls and polyphenols from grape seeds, and probiotics, such as Lactobacilli, Bifidobacteria, yeasts and the like.

Xyloglucans can be present in the invention's compositions in a wide range of concentrations that depends on the type of composition and on the therapeutic indication for which they are intended.

In one embodiment, a suitable range of concentration of xyloglucan in a single administrative dose is between about 0.5 mg/dose and 2000 mg/dose, preferably between 1 mg/dose and 500 mg/dose.

Xyloglucan may be administered orally at doses between about 0.5 mg/dose and about 200 mg/dose, preferably between about 10 mg/dose and about 100 mg/dose. The total daily dose of xyloglucan can be about 400 mg to about 800 mg.

The protein, in particular gelatin, may be administered orally at doses between about 10 mg/dose and about 3000 mg/dose, preferably between about 50 mg/dose and about 500 mg/dose.

The invention's compositions are useful for the treatment and prevention of intestinal disorders and in any case of disorders that originate in the intestinal system and are transferred to other systems, such as the genitourinary system. In fact, it is known that Gram-negative bacteria present in the intestine, in particular Escherichia coli, can proliferate in this organ and migrate to the urinary tract where they cause 90% of genitourinary infections such as cystitis, cystopyelitis and the like.

In particular, the invention's compositions are useful for preventing the proliferation of pathogens in the intestinal tract and their transfer to other systems of the human body through intestinal tight junctions, as well as for the protection of the intestinal mucosa from chemical or physical agents that can reduce the functionality and natural regeneration of the intestinal epithelium and for the reduction of the paracellular flux of pathogens through the intestinal walls.

The invention's compositions have also proven useful for the prevention and treatment of damage to the intestinal mucosa and consequent inflammatory phenomena such as diverticulosis and the early stages of diverticulitis; for the treatment of symptoms resulting from food allergies (such as intolerance to lactose, gluten, etc.); for the prevention and treatment of digestion disorders (production of gas, bloating, borborygmi (“stomach rumble”), flatulence); for the prevention and treatment of damage to the intestinal mucosa deriving from local inflammatory phenomena, of both a temporary and chronic origin, and in particular for the treatment of Crohn's disease, ulcerative colitis or irritable bowel syndrome (IBS).

The invention's compositions can be advantageously used for the treatment of diarrhoea in combination with electrolytes for oral rehydration, as mucomimetic agents or to inhibit the adhesion of bacteria to the mucosa and the subsequent proliferation that leads to dysbacteriosis, optionally in combination with probiotics or tyndallized bacteria. The invention's compositions are particularly useful for the prevention and treatment of traveller's diarrhoea.

Furthermore, the invention's compositions effectively protect the mucosa and decrease the adhesion of several pathogens, for example gas-producing bacteria, to it. It is expected that mucosal protection would be achievable in the presence of many different pathogenic organisms including bacteria, for example, E. coli, Vibrio cholerae, Clostridium difficile, or other intestinal flora.

The following examples illustrate the invention in greater detail.

### EXAMPLE 1

Composition for the prevention and the treatment of diarrhoea; single dose sachet:

| | |
|---|---|
| Xyloglucan | 0.100 g |
| Gelatin | 0.050 g |
| Inulin | 1.650 g |
| Maltodextrin | 1.195 g |
| Stevioside (Stevia) | 0.015 g |
| Tutti frutti flavour (Firmenich) | 0.015 g |
| E160 colouring (a) (beta-carotene) | 0.025 g |

### EXAMPLE 2

Composition for the prevention and the treatment of diarrhoea; hard capsule:

| | |
|---|---|
| Xyloglucan | 0.1 g |
| Gelatin | 3.0 g |
| Matricaria dry extract | 0.026 g |
| Pectin | 0.050 g |
| Dimethicone | 0.020 g |
| Kaolin | 0.020 g |
| Magnesium stearate | 0.080 g |

### EXAMPLE 3

Composition for the prevention and treatment of diarrhoea, tablet:

| | |
|---|---|
| Xyloglucan | 0.1 g |
| Pea protein | 0.5 g |
| Lactose | 0.063 g |
| Colloidal anhydrous silica | 0.002 g |
| Microcrystalline cellulose | 0.030 g |
| Magnesium stearate | 0.003 g |

### EXAMPLE 4

Biological tests; Mucosal protection with reduction of paracellular flux.

E. coli invasion method was used using the CacoGoblet® (Admecell, Alameda, Calif., USA) commercial kit. According to a first protocol, the Caco-2 cells were pre-treated using xyloglucan at the concentration of 5 mg/ml for 1, 4 or 24 hours. The cells were thus inoculated for 30 minutes using E. coli (1.26×10⁸ CFU/well). Then, there followed the determination of the para-cellular flow and the permeability by measuring the passage of the Lucifer Yellow colouring agent after each exposure to the treatment using xyloglucan at 1, 4 and 24 hrs. This assay allowed evaluating the integrity of the cell junctions in the presence of the substance under scrutiny.

The transepithelial electrical resistance (TEER) which provides a direct measurement of the barrier function and which is a further parameter of the wholeness of barrier at the narrow junction level was also evaluated.

The same measurements of passage Lucifer Yellow colouring agent and the TEER were also carried out using a treatment protocol, in which the Caco-2 cells were first inoculated using E. coli according to the previously described methods and thus treated after 1, 4 and 24 hours using xyloglucan.

Upon testing the passage of Lucifer Yellow colouring agent in the prevention protocol, the treatment using xyloglucan caused a marked reduction of the paracellular flux at all considered times, from 8.79% of the initial value after challenge using E. coli to 3.84, 3.45 and 3.95% at 1, 4 and 24 hrs, respectively. Xyloglucan also revealed to be capable of reducing the adhesion of E. coli on the intestinal mucosa.

In the treatment protocol, xyloglucan was capable of reducing the para-cellular flow from 8.11 to 3.44 after 1 hr and from 8.28% to 3.01% after 4 hrs. The recovery percentage of the wholeness of the barrier, measured by the TEER values, increased from 0 to 28% after one hour rising up to 81% after 4 hours. The wholeness of barrier could still be observed after 24 hours.

The results clearly reveal the capacity of xyloglucan to act as a filmogenic agent capable of restoring the functionality of barrier and reducing permeability both in the post-infection treatment and in the prevention model. Xyloglucan is thus efficient at protecting the intestinal mucosa from adhesion and damage caused by E. coli and protecting the structure of the narrow cell junctions with long-term efficiency.

### EXAMPLE 5

Biological tests: protection from the secretion of intestinal water induced by cholera toxin in rats using a Xyloglucan and Gelatin combination (in accordance with Botella, et al., Peptides (1993) 14(2): 297-301; and Velazquez, et al., J. Ethnopharmacol. (2012) 143(2): 716-719).

Animal preparation. After on overnight fast, rats were anesthetized with pentobarbitone sodium (60 mg/kg sc) and anaesthesia maintained by re-administration of pentobarbitone (15 mg/kg/h). Anesthetized animals were maintained at 35° C. by placing them on a heated pad and the trachea was cannulated to prevent any airway obstruction. A midline laparotomy was made to expose the intestine and 10 cm long jejunal segments (4) were isolated and rinsed with hot saline then replaced with 1-5 ml of saline (NaCl 0.9%) containing 10 μg of cholera toxin with or without local administration of the test-compounds. The intestinal segments were replaced into the peritoneal cavity and the abdomen closed. Two hours later, the animals were sacrificed and each isolated segment weighted before and after emptying them. Experimental oral treatments were delivered either 6 or 12 hours before CT intra-loop administration.

Measurement of water secretion. Immediately after sacrifice (2 h), the closed intestinal segments were weighed before and after elimination of contents, these measurements demonstrating the level of water secretion expressed in grams as the difference between full and empty jejunal loops.

Xyloglucan (XG) was given orally, alone or in combination with gelatin or co-administered with cholera toxin (CT) into isolated jejunal loops in anesthetized rats. Evaluation of CT-induced water secretion was performed 2 hours later. Four groups of Wistar rats (200-220 g) were treated as follows: group1 as the control group (vehicle) with no CT+1 ml of saline; group 2 as the positive group (vehicle+CT in 1 ml saline); group 3 treated orally with 12.5 mg/kg of xyloglucans (XG 12.5 mg/kg/PO at t=−6 h) then+CT in 1 ml saline; group 4 treated orally with a combination of 12.5 mg/kg of xyloglucans and 125 mg/kg of gelatin (XG 12.5 mg/kg+P 125 mg/kg/PO at t=−6 h) then+CT in 1 ml saline). Six hours after treatment or oral administration, the groups of animals were treated with cholera toxin (CT) at a dose of 10 μg/ml.

Two hours after treatment with the toxin, the water content of the intestinal loop was measured.

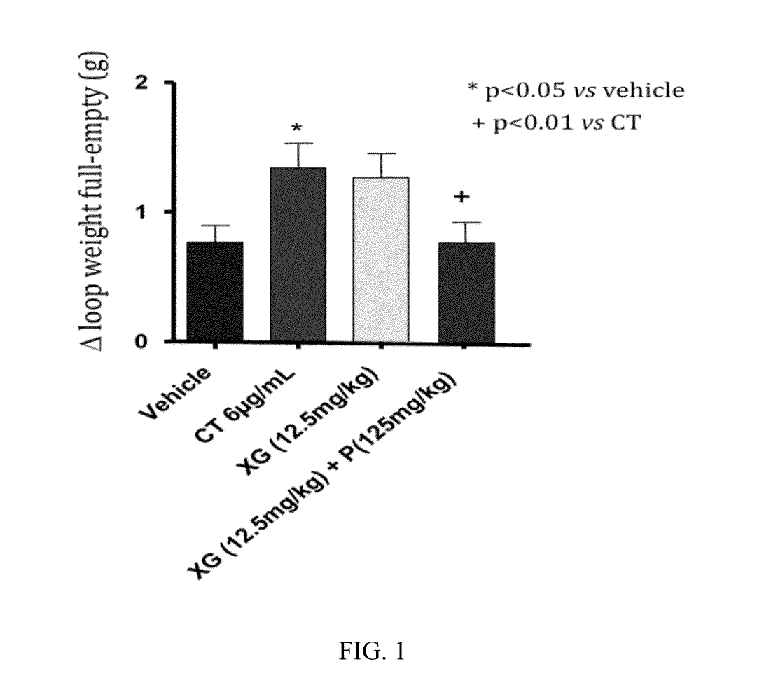

The results obtained, reported in FIG. 1, demonstrate that xyloglucans alone did not reduce water secretion, while the effects of the combination of xyloglucans and gelatin (P) were instead statistically significant. The combination of xyloglucan (XG) and gelatin (P) showed an intestinal water content reduced almost to baseline, even after CT challenge, and a reduction of greater than 30%, or more, compared to treatment with xyloglucan alone.

### EXAMPLE 6

Biological tests: protection from the secretion of intestinal water induced by cholera toxin in rats using a Xyloglucan (XG) and Gelatin (Gel) combination—Influence of gelatin dose. Statistics: For water weight into the loop, data in grams were expressed as mean±SD. Comparisons between the different treatments were performed either by parametric Student's t test after ANOVA or using a non parametric test “Mann Whitney test” if required. The criterion for statistical significance was set up at p<0.05.

The procedure of Example 5 was repeated.

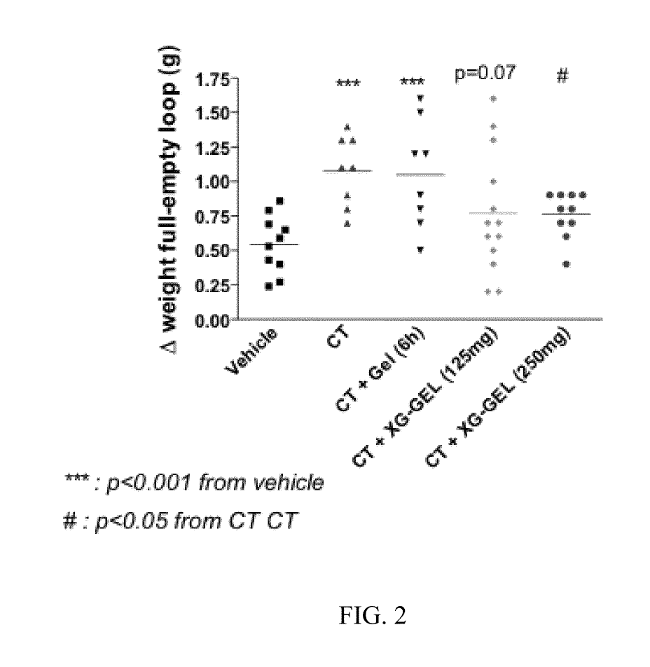

On basal conditions, the mean weight of intra-loop water after 2 hours was: 0.41±0.11 g/loop (n=10). Addition of CT to the loop, 6 hours after oral vehicle, increased this volume to 1.04±0.32 g/loop. When the animals were previously orally treated 6 hours before with gelatin alone, the weight of water present in the loop 2 hours after CT was not significantly different from vehicle (1.01±0.39 g/loop) (FIG. 2).

When CT challenge was performed 12 hours after oral gavage with xyloglucan (12.5 mg/kg)+gelatin (125 mg/kg), the weight of water was reduced to 0.65±0.37 g/loop but the difference did not reach the significance (P=0.07). In contrast, when the animals received 12 hours earlier xyloglucan (12.5 mg/kg)+gelatin (250 mg/kg), after the CT challenge the weight of water found into the loop was significantly (P<0.05) reduced (0.75±0.16 g/loop) with an homogenization of the values (FIG. 2).

In this example, it was confirmed that gelatin orally administered alone, 6 hours before CT was inactive to prevent CT-induced secretion. Combined with previous reported data, this result reinforces the hypothesis that gelatin, having no effect per se, is able to prolong the protective activity of XG to 6 hours. In addition, it has been shown herein that this protective action of XG may be extended to 12 hours by increasing to 250/mg/kg the added gelatin. However it was also observed that, in a lower amount (125 mg/kg), gelatin extended in part, the protective effect of XG to 12 hours (even though significance was low).

Note that 250/mg/kg added gelatin corresponds to 25 mg/kg in humans when adjusted on the basis of the metabolic weight (P^{0.75}).

In accordance with the results of Examples 5 and 6, it is believed that combinations of xyloglucan and gelatin (or other suitable protein) can also be used to treat or prevent diarrhoea, or symptoms thereof, in a mammal, e.g., a human.

### EXAMPLE 7

Clinical study in humans.

A controlled parallel multi-centre clinical study was performed by administrating, to patients with acute diarrhoea (N=150, men and women, mean age 47, in 3 equal groups of 50 subjects), the combination of the invention (xyloglucans 800 mg/day and gelatin 2000 mg/day; namely: 2 capsules were administered every 6 hrs at 100 mg xyloglucan and 250 mg gelatin per capsule), the probiotic S. boulardii (at a daily dose of 7×10⁹ cells/dose), or diosmectite (Smecta®, 3 3-g sachets/day). The rapidity of the establishment of clinical efficacy (reduction in the duration of acute diarrhoea and its related symptoms) was evaluated in the three groups. The symptoms considered were nausea, vomiting, flatulence, abdominal plan and bowel movements. The symptoms decreased in all three groups.

The invention's combination resulted in a more rapid action, blocking diarrhoea within 24 hours of the start of the treatment, particularly at 6 hours from the first dose. The invention's combination caused a more rapid decrease in bowel movements according to types 6 and 7 of the Bristol scale with a 60% reduction as compared to 34% and 39% for diosmectite and S. Boulardii, respectively. After 48 hours all three groups had an almost complete resolution of this type of bowel movement. Abdominal pain was monitored for the entire duration of the treatment. The patients had no vomiting after 48 and 72 hours.

The invention's combination was therefore the most rapid in stopping bowel movements.

The compositions may be administered by any route, including but not limited to oral, sublingual, buccal, ocular, pulmonary, rectal, and parenteral administration, or as an oral or nasal spray (e.g. inhalation of nebulized vapors, droplets, or solid particles).

The pharmaceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user. Useful excipients include microcrystalline cellulose, magnesium stearate, calcium stearate, any acceptable sugar (e.g., mannitol, xylitol), and the like.

Whereas, the present invention has been described in relation to certain embodiments thereof, and many details have been put forth in its illustration, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A composition for use in the prevention and treatment of intestinal disorders or of disorders originating in the intestinal system and transferred to other systems, said composition comprising xyloglucans or extracts containing xyloglucans, and at least one animal or vegetable protein.

2. The composition according to claim 1, wherein the disorder originates in the gastro-intestinal system and is transferred to the genitourinary system.

3. The composition according to claim 1, wherein the intestinal disorder is diarrhoea.

4. The composition according to claim 1, wherein said preventive treatment is selected from the group consisting of (a) the treatment of damage of the intestinal mucosa and of the consequent inflammatory conditions selected from diverticulosis or early stages of diverticulitis; (b) the treatment of symptoms consequent to alimentary allergies; (c) the prevention and treatment of digestive disorders; and (d) the prevention and treatment of damages of the intestinal mucosa deriving from local inflammatory conditions, of temporary or chronic origin.

5. The composition according to claim 4, wherein said composition is used for the prevention and treatment of a disorder selected from Crohn's disease, ulcerative colitis, Irritable Bowel Syndrome (IBS), diverticolosis, early stages of diverticulitis, gluten or lactose intolerance, stomach rumble, meteorism, flatulence, or traveler's diarrhoea.

6. The composition according to claim 1, wherein the at least one animal or vegetable protein is selected from the group consisting of gelatin, albumin, ovalbumin, casein, pea protein, soy protein, and mixtures or blends thereof.

7. The composition according to claim 6, wherein the at least one animal or vegetable protein is gelatin.

8. The composition according to claim 7, wherein the weight ratio between xyloglucan and gelatin is between about 1:0.5 and about 1:30.

9. A composition according to claim 1, wherein said composition further comprises one or more active ingredients selected from the group consisting of antibiotics, antiperistaltic agents, steroidal or non-steroidal anti-inflammatory agents, compounds for the treatment of gastrointestinal bloating, simethicone, mesalazine, sucralfate, pectins, chitosan, hyaluronic acid, Guar gum, xanthan gum, celluloses, hemicelluloses, hydroxypropyl cellulose, carrageenans, carbomers, ferulic acid; polyphenols, probiotics, and electrolytes.

10. A method for prevention or treatment of intestinal disorders or of disorders originating in the intestinal system and transferred to other systems, said method comprising the steps of:
(a) providing a composition comprising xyloglucans or extracts containing xyloglucans, and at least one animal or vegetable protein; and
(b) administering an effective amount of the composition to a mammal in need thereof, said mammal having an intestinal disorder or a disorder originating in the gastro-intestinal system and transferred to other systems.

11. The method of claim 10, wherein the disorder originates in the gastro-intestinal system and is transferred to the genitourinary system.

12. The method according to claim 11, wherein the at least one animal or vegetable protein is selected from the group consisting of gelatin, albumin, ovalbumin, casein, pea protein, soy protein, and mixtures or blends thereof.

13. The method according to claim 12, wherein the at least one animal or vegetable protein is gelatin.

14. The method according to claim 13, wherein the weight ratio between xyloglucan and gelatin is between about 1:0.5 and about 1:30.

15. The method according to claim 10, wherein composition is administered orally.

16. The method according to claim 15, wherein the oral daily dose of xyloglucan is between about 0.5 mg and about 800 mg, and the oral daily dose of gelatin is between about 10 mg and about 3000 mg.

17. The method according to claim 10, wherein said composition further comprises one or more active ingredients selected from the group consisting of antibiotics, antiperistaltic agents, steroidal or non-steroidal anti-inflammatory agents, compounds for the treatment of gastrointestinal bloating, simethicone, mesalazine, sucralfate, pectins, chitosan, hyaluronic acid, Guar gum, xanthan gum, celluloses, hemicelluloses, hydroxypropyl cellulose, carrageenans, carbomers, ferulic acid; polyphenols, probiotics, and electrolytes.

18. The method according to claim 10, wherein said disorder is diarrhoea.

19. The method according to claim 10, wherein the mammal is a human.

20. A method for the prevention of diarrhoea, comprising the steps of:
(a) providing a composition comprising xyloglucans or extracts containing xyloglucans, and a gelatin; and
(b) orally administering an effective amount of the composition to a mammal in need thereof, whereby diarrhoea, or symptoms thereof, are prevented.
